(19) United States
(12) Patent Application Publication
(10) Pub. No.: US 2011/0251529 A1
(43) Pub. Date: Oct. 13, 2011

(54) REAL-TIME ABLATION MONITORING FOR DESIRED LESION SIZE

(75) Inventors: John Petruzzello, Carmel, NY (US); Ajay Anand, Fishkill, NY (US); Shiwei Zhou, Yorktown Heights, NY (US)

(73) Assignee: KONINKLIJKE PHILIPS ELECTRONICS N.V., EINDHOVEN (NL)

(21) Appl. No.: 13/140,040

(22) PCT Filed: Dec. 3, 2009
(86) PCT No.: PCT/IB2009/055490

§ 371 Date: Jun. 16, 2011

Related U.S. Application Data
(60) Provisional application No. 61/139,868, filed on Dec. 22, 2008.

(51) Int. Cl. A61N 7/02

(52) U.S. Cl. 601/3

(57) ABSTRACT

An ablation control device (110) configured for halting, in real time, ablation of body tissue at a current ablation point to achieve a predetermined lesion size upon halting includes a control section (120) configured for registering, with a characteristic curve, one or more values and halting the ablation based on the registering. The value or values are obtained from monitoring (115), for the current ablation point, displacement caused by force applied to the body tissue. In one embodiment, halting is performed upon detecting, by the monitoring and after a peak value of the monitored displacement has occurred, an endpoint value of the monitored displacement. In another embodiment, the end-point value is determined prior to the detecting, and the determining is performed by the registering.

### FIELD OF THE INVENTION

The present invention relates to ablation control, and more particularly to real-time control to achieve a predetermined lesion size.

### BACKGROUND OF THE INVENTION

Tumor ablation therapy using high intensity focused ultrasound (HIFU) has been studied for many years and is just making its way into the United States market and clinical trials.

A tumor, such as a cancer, can be medically treated by surgery and/or chemotherapy. Ablation therapy offers a less intrusive alternative. The ablation may be effected through various alternatives, such as by heating (e.g., radio frequency (RF) ablation, high intensity focused ultrasound (HIFU) ablation, microwave, and laser), freezing (e.g., cryogenic ablation) or chemical action.

HIFU is non-intrusive, in that the thermal energy is applied from outside the body to focus on the tumor, but the energy is not concentrated enough to harm the patient's skin or more internal tissue before it concentrates on the targeted tumor.

Thermal ablation, such as HIFU ablation, raises the temperature at the focal point until the tumor, which may be malignant, is necrosed, i.e., killed, at that ablation point. The necrosed body tissue is known as a lesion. The procedure then moves to another ablation point, and continues point by point until the entire tumor is ablated.

The ablation is guided according to images of the area undergoing treatment. Imaging may be in the form of ultrasound, magnetic resonance imaging (MRI), or x-ray imaging such as fluoroscopy.

MRI is employed for guiding HIFU in ablation, but is expensive. The expense may confine use of this method to research centers worldwide. Also, there exists the potential problem of thermal ablation equipment being MR-compatible.

Acoustic radiation force, by means of ultrasound, has been proposed for monitoring HIFU ablation.

An ultrasound wave imparts to the targeted body tissue a “push” that concentrates at the focal point of the wave. Imaging data before and after the push can reveal information on the nature of the body tissue subjected to the push.

More particularly, tissue necrosed by HIFU therapy, or by other means, at a particular location becomes, at some point, stiffer than untreated tissue. Accordingly, for the same amount of pushing force, less of an axial displacement occurs. The push and subsequent tracking can detect the lessened displacement, and can therefore be used to detect the existence a lesion formed by ablation.

Lizzi et. al. (“Lizzi”) predicts the use of displacements due to radiation force in real-time HIFU ablation monitoring. F. Lizzi, R. Muratore, C. Deng, J. A. Ketterling, S. K. Alam, S. Mikaelian and A. Kalisz, Ultrasound in Med. & Biol. Vol. 29, No. 11, 1593-1605 (2003).

The Lizzi study proposes that the therapy could be continued until it results in a predetermined alteration in motion characteristics in reaction to pushing.

### SUMMARY OF THE INVENTION

In an aspect of the present invention, it is proposed that conceptualization and implementation of a more fully satisfactory ablation monitoring methodology is needed.

The present invention is directed to addressing the limitations of the prior art in the monitoring of ablation, by providing realization of an accurate, fast, low-cost, simple and convenient technique.

State-of-the-art MRI methods for monitoring HIFU ablation treatment based on temperature are accurate, but require the use of a costly MR suite.

The state of the art in ultrasound guided HIFU (USgHIFU) therapy is to assess the extent of the lesion formed, ablation point by ablation point, after the therapy has been applied.

The time expended in this assessing lengthens the duration of the ablation procedure.

In addition, a typical method is to enter an ablation intensity and a time duration, and then to perform the ablation at the ablation point. However, the instant inventors have observed that treatment time is not a good indicator of lesion size. Thus, the need exists in such a procedure to assess lesion size (and ensure that the desired lesion size has been achieved as per the treatment plan) before moving the therapy focus to the next ablation point.

Furthermore, since the ultrasound solutions that are being used today are not sufficiently accurate in predicting dosage (i.e., duration of HIFU application at the current intensity), the approach is to overdose during treatment to assure necrosis of the entire area.

The Lizzi study predicts the use of acoustic radiation force, an ultrasound technique, in real-time monitoring of HIFU, and the termination of HIFU based on a predetermined alteration of motion characteristics.

However, the Lizzi study does not specify what particular alteration would prudently serve as an indication of when therapy is to be terminated, or when and how the determining of the predetermined alteration is accomplished.

It would be advantageous to have a reliable indicator of when therapy should be halted, one that allows real-time ablation to automatically proceed reliably.

To better address one or more of these concerns, and in accordance with the present invention, halting ablation of body tissue at a current ablation point to achieve a predetermined lesion size involves monitoring, in real time, for the current ablation point, displacement in reaction to force applied to the body tissue. One or more displacement values obtained by the monitoring of the displacement are registered with a characteristic curve. Halting, in real time, the ablation at the current ablation point is based on the registering. The predetermined lesion size is achieved upon halting.

In a further aspect, the halting is performed upon detecting, by the monitoring and after a peak value of the monitored displacement has occurred, an endpoint value of the monitored displacement.

In a yet further aspect, the endpoint value is determined prior to the detecting, and the determining is performed by the registering.

In an additional aspect, the determining of the endpoint value is enabled upon the registering with the characteristic curve.

In yet an additional aspect, ablation at the ablation point is performed in push-therapy cycles that have a monitoring portion and a therapy portion. The determining of the endpoint value is enabled as a result of the first cycle.

In another aspect, the determining of the endpoint value entails fitting a curve to normalized displacement differences for corresponding observed lesion sizes. In this context, a normalized displacement difference is, for a corresponding one of lesions whose size has been observed, a difference between a normalized peak displacement and a normalized endpoint displacement. Here, the endpoint displacement subject to normalization occurs temporally after the peak displacement subject to normalization.

In one further aspect, the characteristic curve is derived from empirical observation. Determining an endpoint value of the monitored displacement further entails evaluation against a histologically determined curve.

In yet another aspect, the applied force is acoustic radiation force. The ablation at the ablation point entails a series of monitoring-therapy cycles. The series is preceded by a push. An initial displacement value arises in reaction to the push. In addition, from the push, the location at which the initial displacement value occurred is detected. Before the series of monitoring-therapy cycles, a therapy focus is aligned, based on the detected location and a pre-designated location coincident with the ablation point.

In an embodiment based on the above aspect, the initial displacement value is a value of a spatially maximum displacement.

In an alternative aspect, the monitoring, registering, and halting are performed automatically and without need for user intervention.

In a particular variation of this latter aspect, these steps are repeated, automatically and without need for user intervention, to advance, repetition by repetition, to a different ablation point. The resulting ablation points are disposed in a specified region of interest, and form a matrix of ablation points that provide coverage over the region of interest.

In accordance with these, and other, aspects of the present invention, more control of dosing is afforded, to prevent under- or over-dosing of tissue. A convenient and economical all-ultrasound implementation is afforded, enabling a much more widespread usage of this type of treatment in the United States and emerging markets.

Details of the novel ablation control are set forth further below, with the aid of the following drawings.

### BRIEF DESCRIPTION OF THE DRAWINGS

FIG. 1 is an exemplary functional diagram of a system in accordance with the present invention;

FIG. 2 is one type of suggested signal timing scheme in accordance with the present invention;

FIG. 3 is an example of a graph illustrating a method by which the therapy focus can be aligned with the targeted ablation point, in accordance with the present invention;

FIG. 4 is one example of a graph of a typical displacement over time and of a quadratic curve fitted to an initial portion of the graph for peak detection, in accordance with the present invention;

FIG. 5 is an exemplary graph of normalized displacement over time, in accordance with the present invention;

FIG. 6 is an example of a graph of lesion diameter versus normalized displacement difference, in accordance with the present invention;

FIG. 7 is a flowchart of an example of preparation and initialization of an ablation control device, in accordance with the present invention; and

FIG. 8 is a flowchart showing exemplary operation of an ablation therapy apparatus, wherein point-to-point movement may be clinician-guided or automatic, in accordance with the present invention.

### DETAILED DESCRIPTION OF EMBODIMENTS

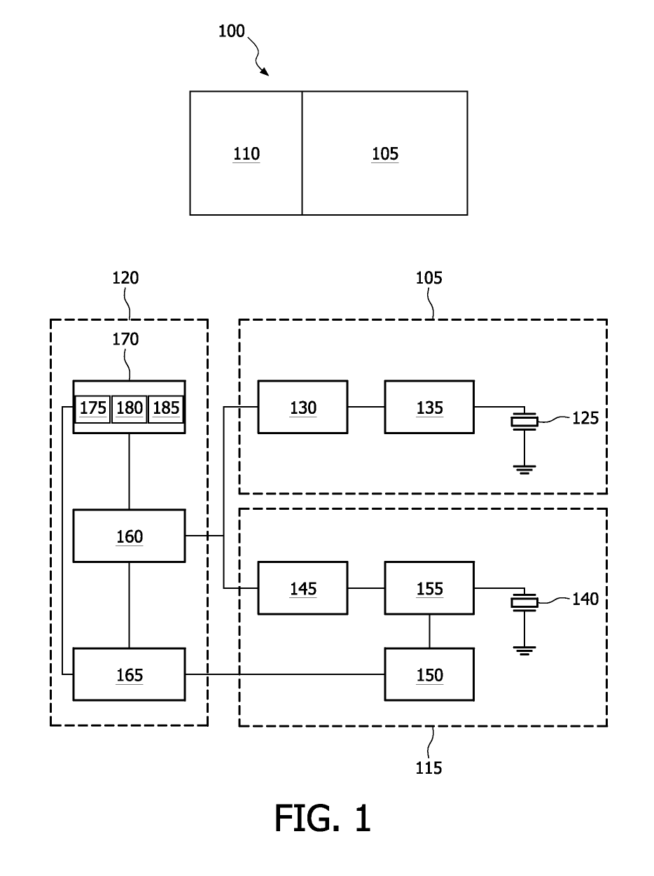

FIG. 1 depicts, by way of illustrative and non-limitative example, a functional diagram of an ablation apparatus 100, comprising, as shown in the upper part of FIG. 1, a therapy section 105 and an ablation control device 110.

As shown in the lower part, in more detail, the ablation control device 110 includes a monitoring section 115 and a control section 120.

The therapy section 105 has a high intensity focused ultrasound (HIFU) transducer 125, connected to an RF (radio frequency) amplifier 130 by means of a matching network 135.

The monitoring section 115 includes an imager transducer 140, connected to a pulser 145 and a receiver 150 by means of a transmit/receive (T/R) switch 155.

The control section 120 comprises an arbitrary waveform generator (AWG) and trigger 160, a digitizer 165, and a processor 170. The processor 170 includes a graphical user interface (GUI) 175, a master signal generator 180 and a motion controller 185 for controlling the positioning of an examining table and the transducers 125, 140. The transducers are housed in a probe to be placed on the patient by computer control or manually. Alternatively the probe may be placed at the end of flexible shaft to be introduced internally, as by the mouth of a patient under anesthesia.

The HIFU transducer 125 focuses ultrasound (which is radio frequency or “RF” energy) to thereby ablate the tumor or other target of ablation. The HIFU transducer 125 is driven by signaling that is amplified in the RF amplifier 130. The amplified signaling passes through the matching network 135 to match impedances of the transducer 125 and the amplifier 130. Although the matching network 135 is provided in the therapy section 105 only, the closeness of impedances between adjacent components may warrant a matching network in the monitoring section 115 too or instead, or may warrant neither section being implemented with a matching network.

The HIFU transducer 125 also delivers ultrasound in the form of an acoustic radiation force imaging (ARFI) push, and receives back the echoes from the ablation subject. The term “ablation subject” hereinafter refers to the medical patient receiving therapy, whether human or animal, or any body tissue such as when testing is conducted.

The imager transducer 140 emits ultrasound to interrogate the extent by which the ARFI push has displaced body tissue. The emitted ultrasound can also be used to assess the extent of the tumor being treated. Although the invention is not limited to separate transducers for pushing and imaging, separate transducers for these two functions allows tracking of the results of a push to closely follow right after the push, to thereby yield more accurate results.

The pulser 145 drives the imager transducer 140, causing the transducer to emit ultrasound toward the ablation subject. The receiver 150 receives back the RF data coming from the ablation subject. The T/R switch 155 switches between these two modes.

The AWG & trigger 160 issues signaling to control the transmission of ultrasound and the reception of the RF data that is echoed back. The AWG may be gated to follow a particular snapshot in time of the heartbeat and/or respiration cycles depending on the location of the in vivo ablation site being subject to ablation.

The digitizer 165 collects the incoming RF data and furnishes it to the GUI 170. The GUI 170 processes the RF data. It also creates images for display on a monitor and for processing to derive displacement data. Associated with the GUI 170 and the monitor are user interface input/output means that may include keys, dials, sliders, trackballs, touch-sensitive screens, cursors and any other known and suitable actuators.

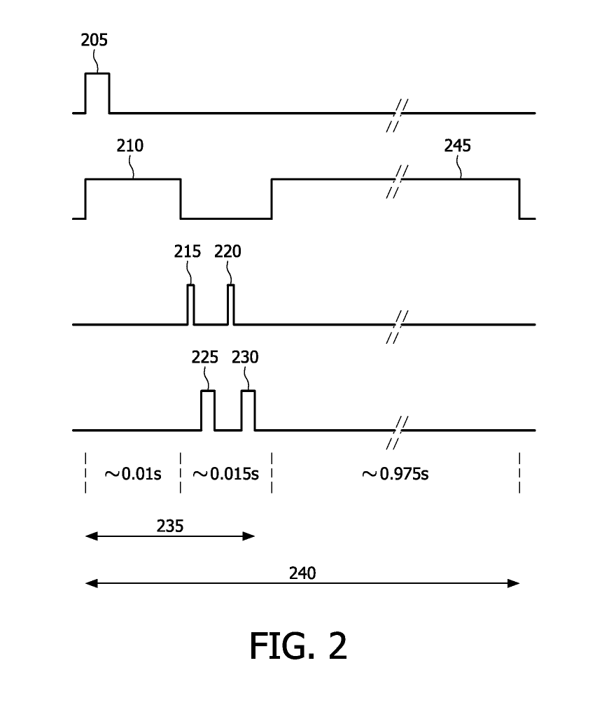

FIG. 2 illustrates one scheme for the synchronization of push, tracking, and therapy pulses in the ablation control device 110. In the exemplary embodiment shown, a master trigger 205 is followed by a push 210 from the HIFU transducer 125. The push duration is set for between 10 and 15 milliseconds (ms), depending on mechanical properties of the tissue to undergo ablation. Following the push 210 are two tracking pulses 215, 220 emanating from the imager transducer 140. The tracking pulses 215, 220 are A-mode pulses, i.e., produced from a single transducer, rather than from an array of transducers, and are employed to perceive structures at different depths along the A-line in the body tissue. Tracking pulse 215 issues immediately after the push 210 to interrogate the strained tissue value. Tracking pulse 220 issues about 12 ms later and represents the relaxed (or equilibrium) tissue value. The digitizer 165 records corresponding return echoes 225, 230 of these two tracking pulses 215, 220 immediately following each of the two pulses. Differences between the RF data retrieved from these two return echoes 225, 230 represent the displacement the body tissue has undergone in reaction to the push 210. This entire sequence is a monitoring portion 235 of a monitoring-therapy cycle 240, and lasts between 20 and 30 ms. The therapy portion 245, during which the HIFU transducer 125 delivers therapy, is much larger, and lasts between 970 and 980 ms. The entire monitoring-therapy cycle 240 lasts for about 1 second, i.e., 1 s.

Other possible timing sequences can be substituted for the one in FIG. 2, such as where the first of the two tracking pulses precedes the push and the second tracking pulse occurs after the push. As in FIG. 2, the spatial position revealed as a result of the first tracking pulse is compared to the spatial position revealed as a result of the second tracking pulse to derive the displacement resulting from the push. As a further example, monitoring may be simultaneous with pushing. Also, the displacement induced may be oscillatory, as with harmonic motion imaging (HMI).

Due to the focused nature of the ultrasound beam being applied in the push 210, displacement is maximal at the focus. However, displacement to lesser extents occurs axially and radially away from the focus. The displacement is affected, over time, by the heat delivered by the therapy ultrasound beam from the HIFU transducer 125.

To take advantage of a larger and more noticeable displacement, and for uniformity in measuring ablation point-to-ablation point, it is desirable to focus the beam delivering the push 210 at the focus of the therapy ultrasound beam (or “therapy focus”) so that the two foci coincide. The two beams emanate from the same HIFU transducer 125. Although the therapy beam is at a higher power than the push beam, the two beams share the same focusing parameters and the same focus (or “focal point”).

The tracking pulses 215, 220 originate from a separate transducer than that producing the push/therapy focus; however, the two transducers 125, 140 are preferably configured in fixed spatial relation and confocal.

To align the therapy focus with the targeted body tissue, the A-mode imaging is used to display the treatment area on-screen before treatment begins.

The clinician may localize (i.e., identify to the system the location of) the upcoming ablation point by pointing to (with a touch-sensitive screen) or navigating to (as by manipulating a mouse) the corresponding point in the image.

Based on this localization, the therapy focus parameters can be adjusted to approximate the specified location.

However, uncertainties in tissue acoustic and thermal properties due to heterogeneities can influence the site of push and therapy. Hence, despite the adjusting of parameters, the push could actually occur at a location that varies somewhat from the location the clinician has indicated.

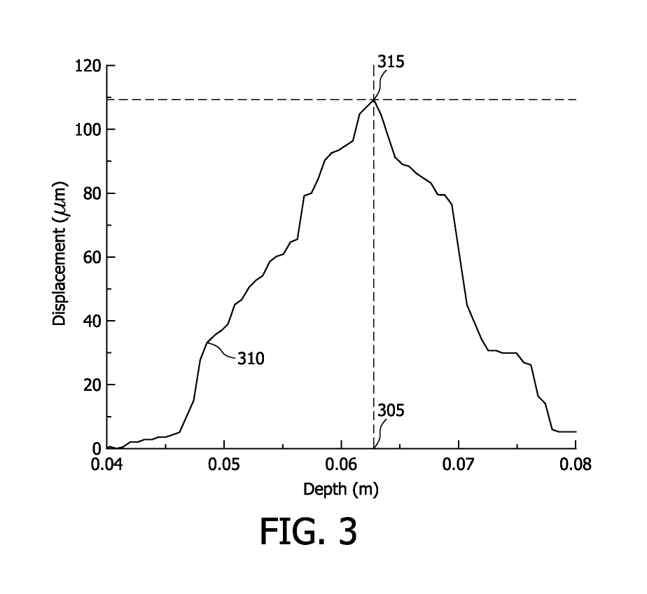

To therefore focus more accurately on the desired location, an additional step is taken after the adjusting of parameters. In particular, the therapy focus is aligned to the specified location based on fed back displacement data, in accordance with an aspect of the present invention, as discussed in more detail with reference to FIG. 3.

FIG. 3 shows a method by which a therapy focus 305 can be accurately located for subsequent alignment with a target ablation point. The FIG. 3 graph represents displacements 310 along an A-line. What is termed an “initial displacement” 315 is the maximum of the displacements 310 along the A-line shown, all resulting from a push 210 of a pre-cycle. Moreover, because the A-line shown is aligned with the push beam, the location of the initial displacement 315 is not only the location of the spatially maximum displacement along the A-line, but an estimate of the spatially maximum displacement in three-dimensional space. Since the push and therapy beams are confocal, the therapy focus 305 coincides with the location of the initial displacement 315. The tracking having now located the initial displacement 315, i.e., the therapy focus 305, all that remains is to align the therapy focus with the location the clinician has indicated on-screen. The latter location is that of the desired, upcoming ablation point. In executing the alignment, the clinician's specification based on the on-screen imaging gained by virtue of the imager transducer 140 is matched to RF data 225, 230 that returns as a result of the tracking pulses 215, 220 emanating from the same transducer.

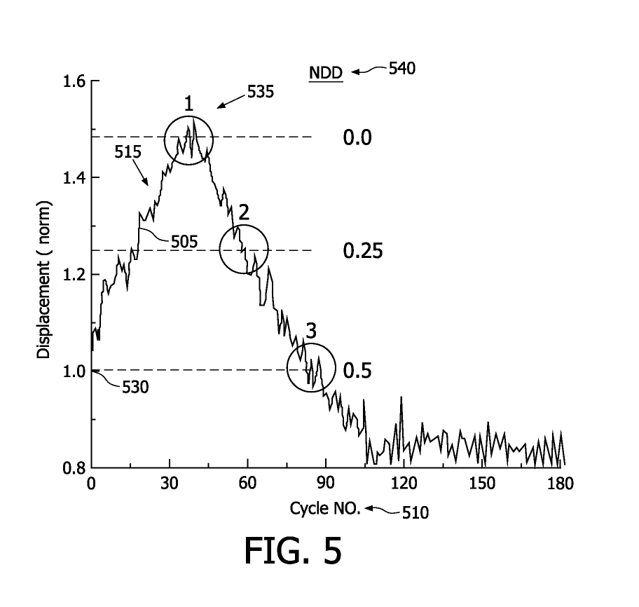

Looking back at the pre-cycle push 210 that has enabled the alignment, the pre-cycle precedes the monitoring-therapy cycles 240, and would not require a therapy portion. The purpose of the pre-cycle push 210 is, as discussed above, to identify the depth into the body tissue at which the initial displacement 315 occurs, which is about 63 mm in the current example, and thereby locate the therapy focus 305. Once the therapy focus 305 is located, it can be aligned with the location specified on-screen, which is the desired, upcoming ablation point. The ablation control device 110 automatically causes this alignment to occur before the next cycle, which is the first of the monitoring-therapy cycles 240. The reason for a pre-cycle, separate from the ensuing monitoring-therapy cycles 240, will be discussed in detail further below in connection with FIG. 5.

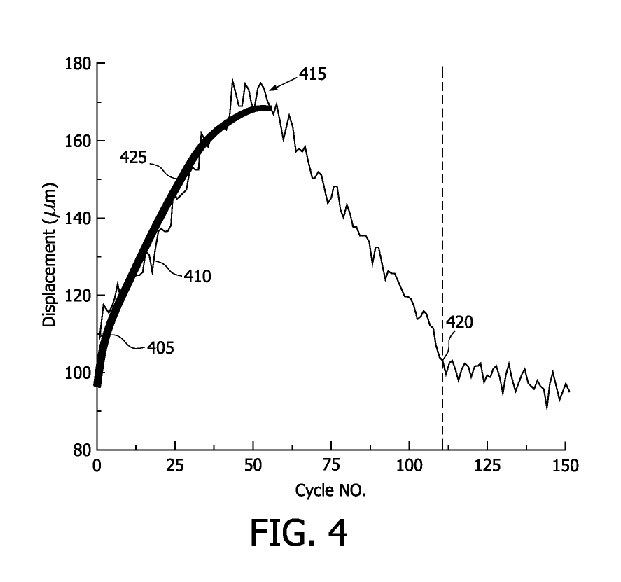

FIG. 4 is an example of a graph of a typical displacement over time and of a quadratic curve fitted to an initial portion of the graph for peak detection. Cycle number zero, in the graph, refers to the start of the monitoring-therapy cycles 240, and occurs after the pre-cycle. In the example of FIG. 4, a starting displacement 405 is shown to be about 110 μm. The starting displacement 405 varies from ablation point to ablation point, individual to individual, and tissue sample to tissue sample, because of the inhomogeneities of the body tissue. Going forward in time, with each successive monitoring-therapy cycle 240, the effect of therapy on the tissue displacement 410 at the therapy focus 305 is obtained. The displacement 410 initially increases over time, due to the applied heat softening the tissue. After some therapy time, the displacement 410 reaches a peak 415 and starts to decrease, indicating that the tissue is becoming stiffer (i.e., upon necrosis). The decrease is observed until the therapy reaches a stopping point in the displacements 410 or “endpoint displacement” 420. After the therapy is turned off, the displacement 410 decrease slows down as the tissue is cooling. However, the effect of temperature on cell necrosis still exists. “Halting ablation of body tissue,” as that expression is used herein, is defined as halting the application, by an ablation apparatus, of mechanical-property-changing energy transfer to body tissue.

A quadratic curve 425 may be fitted to the displacements 410 in real time to detect the peak 415. The peak 415 is detected when the slope of the quadratic curve 425 becomes zero and starts to turn negative. The peak 415 may be estimated by averaging displacement 410 measurements, e.g., for five cycles, within an interval around the zero slope point. A reason for detecting the peak 415 will be discussed in detail below in connection with FIG. 5.

FIG. 5 is an exemplary graph of normalized displacement 505 over time, or, more specifically, according to cycle number 510. The FIG. 5 graph, termed hereinafter a characteristic curve 515, can be derived from the displacement graph of FIG. 4 by dividing each displacement 410 by the starting displacement 405. The characteristic curve 515 may also be a combination, such as an average, of a number of such derived curves, based on empirical observation at different ablation points. Due to the above-noted inhomogeneities of body tissue, the FIG. 5 time scale (of cycle numbers 510) may shrink or expand, depending on the ablation point, individual or tissue sample. Thus, the time rate of normalized displacement is variable. However, the shape of the characteristic curve 515 remains constant for a given type of body tissue, e.g., liver, breast, heart. By implication, once a point on the characteristic curve 515 has been identified, all points are identified. This is significant, because some of the points on the characteristic curve 515 are associated with specific lesion sizes. Thus, the ability to identify that an ongoing ablation at an ablation point has reached a specific point on the characteristic curve 515 can lead to an accurate prediction of when to halt the ablation to achieve a desired lesion size. The word “characteristic” in the term “characteristic curve” as used herein refers to a distinguishing feature or attribute. The distinguishing feature or attribute may pertain to body tissue.

During the current ablation, the pre-normalized displacements 410 are available in real time. A technique in accordance with the present invention is to register one or more displacements 410 with the associated normalized displacement(s) 505 of the characteristic curve 515.

Two landmark points on the characteristic curve 515 are the normalized starting displacement 530, which by convention is set to unity, and the normalized peak displacement 535.

The associated pre-normalized displacements are, respectively, the starting displacement 405 and the peak displacement 415.

The starting displacement 405 is immediately preceded by the pre-cycle. As mentioned further above, the pre-cycle need not have a therapy portion. In fact, it is preferable that it not have one. This is because, due to inhomogeneities in the body tissue, the heating during that approximately one second does not result in a reliable displacement value at commencement of the monitoring-therapy cycles 240. A reliable displacement value 410 at that commencement time is desirable, in order to use the displacement value as a basis for predicting when to halt ablation at the current ablation point. Notably, since, by the commencement time, alignment to the current ablation point has occurred, prediction is appropriately based upon data derived from the current ablation point. Moreover, if the pre-cycle omits a therapy portion 245, the above-noted thermal inhomogeneity effect is avoided. Consequently, the displacement value of the first of the monitoring-therapy cycles, i.e., the starting displacement 405, may be relied upon, in accordance with the present invention, in predicting a stopping point for the ablation at the current ablation point. More specifically, the starting displacement 405 may be registered to the starting normalized displacement 530. The registration allows, by means of the characteristic curve 515, the starting displacement 405 to be utilized in predicting when, displacement-wise, ablation should be halted to achieve a predetermined lesion size upon halting. The starting displacement 405 is accordingly one of the values that can serve as what is termed hereinafter a therapy-progress-rate-independent (TPRI) registration point, as discussed in detail further below.

The peak displacement 415 occurs simultaneously with the normalized peak displacement 535. Accordingly, the peak displacement 415 can, like the starting displacement 405, serve as a TPRI registration point.

For its effectiveness as a predictor of lesion size, registration of the TPRI registration point to the characteristic curve 515 relies on a functional relationship between decrements in normalized displacement 505 and empirical values of lesion size. For this purpose, a normalized displacement difference (NDD) 540 is defined as the difference between the normalized peak displacement 535 and an endpoint of the normalized displacement 505. NDD 540 values of 0, 0.25 and 0.5 are shown in FIG. 5. Thus, for example, with an NDD equal to 0, the normalized peak displacement 535 and the normalized endpoint displacement 505 are the same, which would imply that the application of ablation energy is halted at peak displacement 415 (or, equivalently, at normalized peak displacement 535). A particular lesion size is associated with each value of the NDD 540.

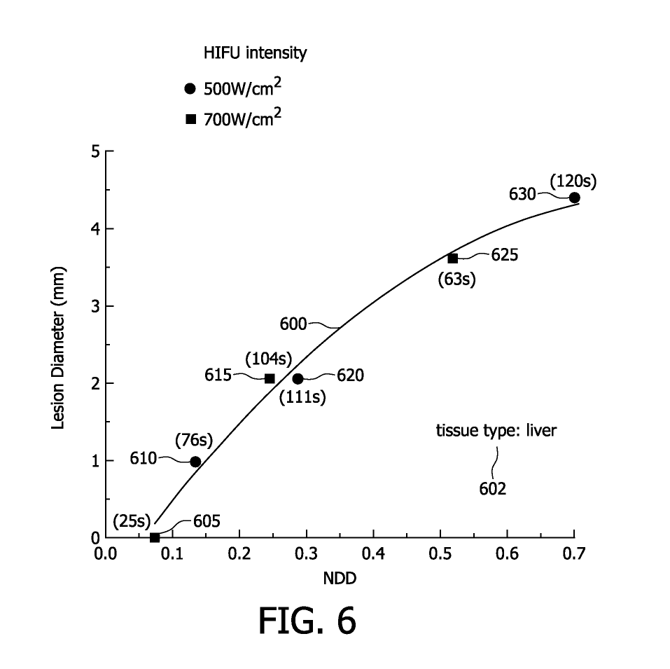

FIG. 6 is an example of a graph 600 of lesion diameter versus NDD 540. Ablation was conducted experimentally on various tissue samples and various sites within a sample. The ablation was halted, and the sample was immediately cooled to stop necrosing. The size of the lesion was measured. The lesion shape depends on the transducer geometry and its acoustic beam characteristics. In the case of HIFU, the lesion shape is commonly ellipsoidal with the major axis along the beam's longitudinal center. The lesion diameter in FIG. 6 accordingly refers to the maximum lesion diameter perpendicular to the beam's longitudinal center. For each measurement, the treatment time, endpoint displacement value 420 and peak displacement value 415 were noted. Based on this actual data, observation points were plotted, relating lesion diameter to NDD 540. FIG. 6 shows some plotted observation points for the tissue type 602, which in this case is liver. It was found that the relationship is described by a second order polynomial fit with good agreement, and that the parameters of the polynomial vary with tissue type. The parameters would also vary with lesion shape, although lesion shape would not typically be varied. It is therefore assumed hereinafter that, when curves are classified by tissue type, there exists no need to further classify by lesion shape. As shown by the different HIFU intensities of the observations 605-630, the fitted function is invariant with treatment intensity. The treatment times for the six samples are listed in parentheses. It can be seen that the treatment time is not a good indicator of lesion size, due to inhomogeneities of the tissue. Observation 615, for example, indicates more treatment time to achieve a smaller lesion size in comparison to observation 625. For observations made for different parts of the same tissue sample or for different tissue samples, lesion sizes have been found not to correlate well with treatment time. Advantageously, methodology of the present invention, as set forth hereinabove and in more detail below, overcomes sensitivity to tissue inhomogeneity.

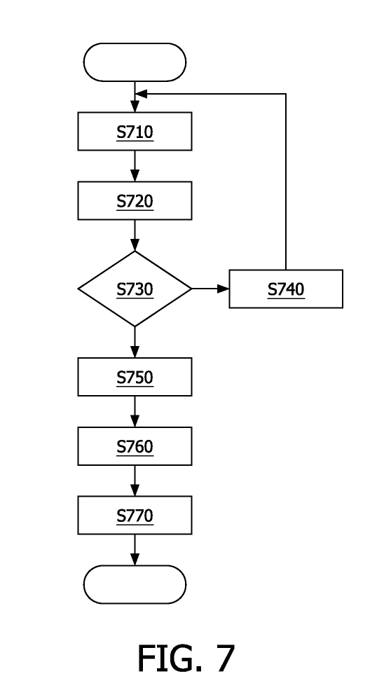

FIG. 7 provides an example of preparation and initialization of the ablation control device 110. Ablation is performed on a particular tissue sample (step S710). Ablation is terminated for the current tissue sample, which is immediately cooled to stop necrosis. Endpoint displacement 420 and peak displacement 415 have been recorded. After histological examination of the lesion formed, the lesion size is recorded (step S720). Query is then made on whether this is the last observation (step S730). If it is not the last observation, a next observation is made, on the current tissue sample or another tissue sample or on another tissue type (step S740). On the other hand, if it is the last observation, the observations are grouped by tissue type (step S750). Fitted curves 600 (or “calibration curves”) are derived by tissue type, using the recorded data and quadratic curve fitting (step S760). The calibration curves 600, each with its identifier of tissue type 602, are sent to the ablation control device 110 . Also, each characteristic curve 515, identified by tissue type, is made available to the ablation control device 110. The characteristic curves 515 have, likewise, been derived from empirical observation, as mentioned above (step S770).

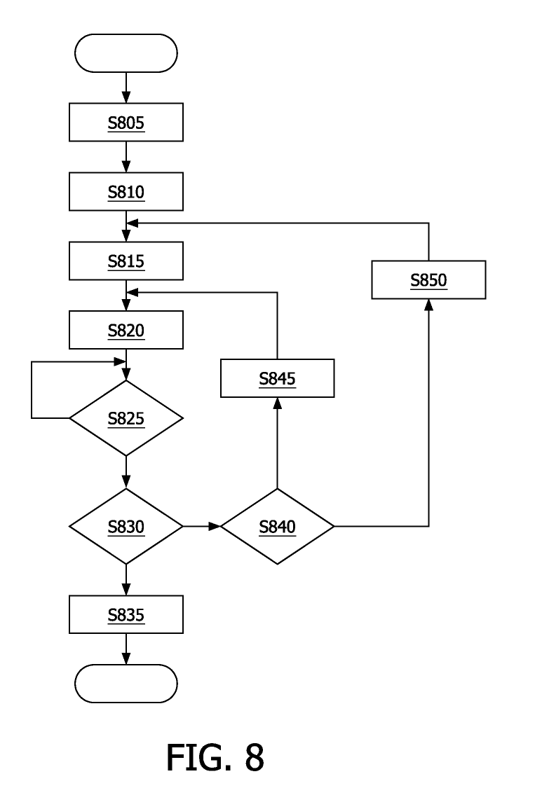

FIG. 8 demonstrates exemplary operation of the ablation apparatus 100, for which point-to-point movement may be clinician-guided or automatic. Upon beginning the ablation procedure, the ultrasound probe is positioned in proximity of the ablation subject and activated. A-mode on-screen imaging (i.e., based on a scan by a single transducer), which may be combined to display as an M-mode scan (by an array of transducers affording multi-dimensional motion in the display), is used by the clinician to define boundaries of the upcoming ablation, as by pointing or navigating on-screen (step S805). The clinician enters the tissue type, and desired lesion size which is then evaluated against the respective calibration curve 600 to yield an NDD 540 (step S810). The order of steps S805 and S810 may be reversed or interspersed. Although the desired lesion size is not yet needed for the formula (1) calculation (shown below), typically the clinician would specify the desired lesion size before beginning ablation at the ablation point. The clinician next points out, on-screen, the current ablation point (step S815). In step S820, the ablation process is commenced at the current ablation point, monitoring of push-induced displacements begins, and one or more TPRI registration point(s) are obtained, in real time, and processed, in real time. The processing involves registering the point(s) (e.g., starting displacement 405, peak displacement 415) to the corresponding point(s) (i.e., normalized starting displacement 530, normalized peak displacement 535) on the appropriate characteristic curve 515. The following formula may be used:

HD=(NPD−NDD)×RP/CP [formula (1)]

where HD stands for the displacement upon which ablation is to be halted;
RP stands for TPRI registration point;
CP stands for the corresponding point of the characteristic (i.e., normalized) curve 515;
NPD stands for normalized peak displacement 535; and
NDD stands for normalized displacement difference 540.

Thus, the determining of the HD, i.e., endpoint displacement 420, is enabled by the registering of the TPRI registration point(s) with the characteristic curve 515. Therefore, for example, if the starting displacement 405 serves as the TPRI registration point, the enabling occurs upon completion of the monitoring portion 235 of the first of the monitoring-therapy cycles 240. Prior to that completion, the starting displacement 405 is not yet known, and therefore cannot be applied as RP in formula (1) shown above.

The quantity RP/CP in formula (1) may be regarded as a normalization factor. When the desired lesion size is evaluated against the calibration curve 600, the NDD 540 is identified. The NDD 540 is subtracted from the NPD 535 to yield the normalized form of the endpoint displacement 420. This normalized form is multiplied by the normalization factor to yield the “de-normalized” endpoint displacement (or HD in formula (1)). If more than one registration point is used, the corresponding normalization factors can be averaged for use in equation (1).

As set forth above, one of the interesting features, according to the present invention, is the registering with the characteristic curve 515 during the ablation procedure at the current ablation point to determine an endpoint displacement 420. Another interesting feature, in accordance with an aspect of the present invention, is that determination of the endpoint value 420 of the monitored displacement entails evaluation against the histologically determined curve 600.

Ablation is halted when the endpoint displacement 420 is detected (step S825). If this is the last ablation point (step S830), the procedure is completed, and a matrix of ablation points around the current ablation point provides coverage over the region of interest, i.e., the tumor or other target area to be ablated (step S835). Otherwise, if this is not the last ablation point (step S830), the next step depends on whether the next ablation point is to be selected manually or automatically (step S840). If selection is to be automatic, the next ablation point serves as the current ablation point (step S845) and processing returns to step S820, whereby processing point to point is performed automatically and without the need for user intervention. If, on the other hand, selection is to be manual, the next ablation point serves as the current ablation point (step S850) and processing returns to step S815.

A systematic method for halting, in real time, ablation of body tissue at a current ablation point to achieve a predetermined lesion size upon halting involves registering, with a characteristic curve 515, one or more values and halting the ablation based on the registering. The value or values are obtained from monitoring, for the current ablation point, displacement 410 caused by force applied to the body tissue. In one embodiment, halting is performed upon detecting, by the monitoring and after a peak value 415 of the monitored displacement has occurred, an endpoint value 420 of the monitored displacement. In another embodiment, the endpoint value 420 is determined prior to the detecting, and the determining is performed by the registering.

In accordance with the present invention, an accurate, fast, low-cost, simple and convenient technique is proposed for halting ablation of body tissue at an ablation point. A convenient and economical all-ultrasound implementation is afforded, enabling a much more widespread usage of this type of treatment in the United States and emerging markets.

HIFU, being an ultrasound method, affords a low-cost all-ultrasound ablation therapy apparatus with features set forth herein above. Nevertheless, any other form of ablation therapy which likewise causes body tissue to undergo a change in mechanical properties is within the intended scope of the present invention, such as by heating (e.g., radio frequency (RF) ablation, high intensity focused ultrasound (HIFU) ablation, microwave, laser), freezing (e.g., cryogenic ablation) or chemical action.

The present invention is not limited to tumor ablation. The alleviation of cardiac arrhythmia, for example, may be accomplished by necrosing a specific line of heart tissue to thereby block an abnormal electrical path through the heart. Such a method may be accomplished using ablation methods of the present invention.

Moreover, although methodology of the present invention can advantageously be applied in providing medical treatment, the scope of the present invention is not so limited. More broadly, techniques of the present invention are directed to placing, and controlling the size of, lesions in body tissue, in vivo, in vitro or ex vivo.

It should be noted that the above-mentioned embodiments illustrate rather than limit the invention, and that those skilled in the art will be able to design many alternative embodiments without departing from the scope of the appended claims. For example, the HIFU transducer 125 may be implemented as a transducer array with separate apertures for pushing and therapy. As a further example, the HIFU transducer 125 and the imager transducer 140 may be replaced with a dual mode transducer for both imaging and therapy. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer having a computer readable medium. The mere fact that certain measures are recited in mutually different dependent claims does not indicate that a combination of these measures cannot be used to advantage.

What is claimed is:

1. An ablation control device (110) configured for halting ablation of body tissue at a current ablation point to achieve a predetermined lesion size, comprising:
a monitoring section (115) configured for monitoring, in real time, for said current ablation point, displacement in reaction to force applied to the body tissue; and
a control section (120) configured for registering (S820), with a characteristic curve (515), one or more displacement values obtained by said monitoring of said displacement and for halting, in real time, said ablation at said ablation point based on said registering, said predetermined lesion size being achieved upon said halting.

2. The ablation control device of claim 1, wherein said halting is performed upon detecting, by said monitoring and after a peak value of the monitored displacement has occurred, an endpoint value (420) of said monitored displacement.

3. The ablation control device of claim 2, wherein said control section is further configured for determining (S820) said endpoint value prior to said detecting, said determining being performed by said registering.

4. The ablation control device of claim 3, wherein said determining is enabled upon said registering (S820).

5. The ablation control device of claim 4, wherein said ablation at said ablation point is performed in push-therapy cycles (240) that have a monitoring portion (235) and a therapy portion (245), the enabling occurring as a result of a first of said cycles.

6. The ablation control device of claim 5, wherein the enabling occurs upon completion of said monitoring portion (235) of said first cycle (S820).

7. The ablation control device of claim 3, wherein said determining is based on histological examination (S720).

8. The ablation control device of claim 3, wherein said determining comprises fitting a curve (S760) to normalized displacement differences (540) for corresponding observed lesion sizes, a normalized displacement difference being, for a corresponding one of lesions whose size has been observed, a difference between a normalized peak displacement and a normalized endpoint displacement, the endpoint displacement subject to normalization occurring temporally after the peak displacement subject to normalization.

9. The ablation control device of claim 8, wherein a time rate (510) of said normalized displacement varies with location of a corresponding ablation point, the fitted curve (600) varying with type of body tissue (602) and being invariant with ablation intensity (605-630).

10. The ablation control device of claim 8, wherein said determining further comprises evaluating (S810) a desired lesion size against the fitted curve.

11. The ablation control device of claim 8, wherein said observed lesion sizes have been histologically determined (S720).

12. The ablation control device of claim 8, wherein the fitted curve is a quadratic function (S760).

13. The ablation control device of claim 1, wherein said characteristic curve is derived from empirical observation (S720), and determining an endpoint value of the monitored displacement further comprises evaluation (S810) against a histologically determined curve (600).

14. The ablation control device of claim 1, wherein the ablation to be halted is high-intensity-focused-ultrasound ablation (125).

15. An ablation apparatus comprising:
the ablation control device of claim 1; and
a therapy section (105) configured for causing said ablation at said ablation point and for applying said force, said ablation control device being further configured for controlling said therapy section.

16. A method for operating an ablation apparatus so as to halt ablation of body tissue at a current ablation point to achieve a predetermined lesion size, said method comprising:
monitoring (S820), in real time, for said current ablation point, displacement in reaction to force applied to the body tissue;
registering (S820), with a characteristic curve, one or more displacement values obtained by said monitoring; and
based upon said registering, halting (S825), in real time, said ablation of body tissue at said ablation point, said predetermined lesion size being achieved upon said halting.

17. The method of claim 16, wherein the applied force comprises acoustic radiation force (235), said ablation at said ablation point entailing a plurality of monitoring-therapy cycles, said plurality being preceded by a push (210), an initial displacement value (315) arising in reaction to said push, said method further comprising:
detecting, from said push, a location (305) at which said initial displacement value occurred; and
aligning, before the plural monitoring-therapy cycles, a therapy focus, said aligning being based on the detected location and a pre-designated location (S815) coincident with said ablation point.

18. The method of claim 17, wherein said initial displacement value is a value of a spatially maximum displacement (315).

19. The method of claim 16, wherein said monitoring, said registering and said halting are performed automatically and without need for user intervention (S845).

20. The method of claim 19, further comprising repeating, automatically and without need for user intervention, said method of claim 19, advancing, repetition by repetition, to, in a specified region of interest around said ablation point, a different ablation point to complete a matrix of ablation points that provides coverage over said region of interest (S835).

21. A computer software product for monitoring ablation of body tissue at a current ablation point to achieve a predetermined lesion size, comprising a computer readable medium embodying a computer program that includes instructions executable by a processor to perform a plurality of acts, said plurality comprising the acts of:
monitoring (S820), in real time, for said current ablation point, displacement in reaction to force applied to the body tissue;
registering (S820), with a characteristic curve, one or more displacement values obtained by said monitoring; and
based upon said registering, halting (S825), in real time, said ablation of body tissue at said ablation point, said predetermined lesion size being achieved upon said halting.
